(19) United States
(12) Patent Application Publication
(10) Pub. No.: US 2004/0268422 A1
(43) Pub. Date: Dec. 30, 2004

(54) Methods for correcting mitotic spindle defects associated with somatic cell nuclear transfer in animals

(76) Inventors: Gerald P. Schatten, Pittsburgh, PA (US); Calvin R. Simerly, Cranberry, PA (US); Christopher S. Navara, Cranberry, PA (US)

Correspondence Address: PRESTON GATES ELLIS & ROUVELAS MEEDS LLP, 1735 NEW YORK AVENUE, NW, SUITE 500, WASHINGTON, DC, 20006, US

(21) Appl. No.: 10/821,200

(22) Filed: Apr. 9, 2004

Related U.S. Application Data
(60) Provisional application No. 60/461,139, filed on Apr. 9, 2003.

(51) Int. Cl. A01K 67/027

(52) U.S. Cl. 800/9; 800/21

(57) ABSTRACT

The present invention is directed to various methodologies to make NT a practical procedure for animals, specifically, primates including human and non-human primates. Furthermore, the methods and molecular components provided by the present invention provide a practical means for producing embryos with desired characteristics. In a specific embodiment, the methodology of the present invention comprises introducing nuclei having desired characteristics along with one or more molecular components into an egg, culturing the egg to produce a viable embryo, transferring the embryo to the oviducts of a female, and producing a cloned animal.

### CROSS REFERENCE TO RELATED APPLICATION

The present application claims the benefit, under 35 U.S.C. § 119, of U.S. Provisional Patent Application Ser. No. 60/461,139, filed 9 Apr. 2003, the contents of which are incorporated herein by reference.

### STATEMENT REGARDING FEDERALLY SPONSORED RESEARCH

This invention was made, at least in part, with U.S. government support under grant numbers NIH R37 HD 12913 and 2 R24 RR013632-06, awarded by NIH. The U.S. government may have certain rights in the invention.

### FIELD OF THE INVENTION

The present invention relates to methods for the clonal propagation of animals, including primates. The present invention also relates to methods for producing embryonic stem cells, transgenic embryonic stem cells, and immune-matched embryonic stem cells from primates, including humans. Furthermore, the present invention provides various methodologies and molecular components that may be used for correcting mitotic spindle defects associated with nuclear transfer.

### BACKGROUND OF THE INVENTION

Identical primates have immeasurable importance for molecular medicine, as well as implications for endangered species preservation and infertility. The lack of genetic variability among cloned animals results in a proportional increase in experimental accuracy, thereby reducing the numbers of animals needed to obtain statistically significant data, with perfect controls for drug, gene therapy, and vaccine trials, as well as diseases and disorders due to aging, environmental, or other influences. The “nature versus nurture” questions regarding the genetic versus environmental, including maternal environment or epigenetic influences on health and behavior may also be answered. Consequently, genetically identical offspring, even with differing birth dates, may be investigated (e.g., in studies such as phenotypic analysis prior to animal production; and in serial transfer of germ line cells (such as male germ cells), Brinster et al., 9 SEMIN. CELL DEV. BIOL. 401-09 (1998)), to address cellular aging beyond the life expectancy of the first offspring; and to test simultaneous retrospective (in the older twin) and prospective therapeutic protocols. Epigenetic investigations may be tested using identical embryos of the present invention implanted serially in the identical surrogate to demonstrate that, for example, low birth weight or other aspects of fetal development may have life-long consequences (Leese et al., 13 HUM. REPROD. 184-202 (1998)), the decrease in the IQ of children is related to maternal hypothyroidism during pregnancy (Haddow et al., 341 N. ENGL. J. MED. 549-55 (1999)), or immunogenetics results in uterine rejection (Gerard et al., 23 NAT. GENET. 199-202 (1999); Clark et al., 41 AM. J. REPROD. IMMUNOL. 5-22 (1999); and Hiby et al., 53 TISSUE ANTIGENS 1-13 (1999)).

The cloning of animals by adult somatic cell nuclear transfers has lead to the creation of sheep (Wilmut et al., 385 NATURE 810-13 (1997)), cattle (Kato et al., 282 SCIENCE 2095-98 (1998)), mice (Wakayama et al., 394 NATURE 369-74 (1998)), pigs, cats, rabbits and goats (Baguisi et al., 17 NATURE BIOTECH. 456-61 (1999)). Among the most compelling scientific rationales for cloning is the production of disease models. Cloned animals as models for disease show great promise because the genetics of each clone are invariable.

Stem cell lines have been produced from human and monkey embryos (Shamblott et al., 95 PROC. NATL. ACAD. SCI. USA 13726-31 (1999) and Thomson et al., 282 SCIENCE 1145-47 (1999)). It is not yet known if stem cells from the fully outbred populations of humans or primates have the full totipotency of those from selected inbred mouse strains with invariable genetics. This can now be evaluated within the context of the present invention, for example, by producing therapeutic stem cells from one multiple, later tested in its identical sibling, and in so doing, learning if stem cells might produce cancers like teratocarcinomas.

Theoretically, somatic cell nuclear transfer (SCNT) has the potential to produce limitless identical offspring; however, genetic chimerism, fetal and neonatal death rates (Wilmut et al., 419 NATURE 583-7 (2002); Humpherys et al., 99 PROC. NATL. ACAD. SCI. USA 12889-94 (2002); Cibelli et al., 20 NAT. BIOTECHNOL. 13-4 (2002); and Kato et al., 282 SCIENCE 2095-8 (1998)), shortened telomeres (Shields et al., 399 NATURE 316-7 (1999)), and inconsistent success rates preclude its immediate usefulness. SCNT in macaques has succeeded with blastomere nuclei (Wolf et al., 60 BIOL. REPROD. 199-204 (1999)), but not yet with adult, fetal, or embryonic stem (ES) cells. These concerns notwithstanding, the contradictions and paradoxes raised by SCNT have stimulated new studies on the molecular regulation of mammalian cloning by SCNT.

FIG. 1 illustrates the manipulations and developmental events that culminate in the somatic nucleus within the activated enucleated oocyte during SCNT. These steps include, but are not limited to, enucleation or metaphase-II arrested meiotic spindle removal, somatic cell selection and preparation, nuclear transfer or intracytoplasmic nuclear injection (ICNI), wound healing and drug recovery from both spindle removal and nuclear introduction, and oocyte activation. There are, however, limitations associated with these steps. For example, meiotic spindle removal in primate oocytes (non-human and human like) has unforeseen consequences on the now enucleated oocyte. Unlike oocytes from domestic species of mice, crucial microtubule motors (kinesins) and centrosome molecules (NuMA) are concentrated almost exclusively on the met-II spindle. Removal of the egg DNA along with this spindle eliminates the majority of these motors and spindle pole proteins so that the SCNT reconstituted oocyte no longer has the ability to form a functional bipolar mitotic spindle at first mitosis.

In addition, there is also a lack of fundamental scientific knowledge related to some of these steps. For instance, somatic cell preparation and selection (Wilmut et al., 385 NATURE 810-3 (1997); Wilmut et al., 419 NATURE, 583-7 (2002); and Wakayama et al., 394 NATURE 369-74 (1998)) has only been investigated in a small number of species, a comparison between electrofusion versus direct injection (Intracytoplasmic nuclear injection (ICNI)) has not been fully investigated, and wound healing after microinjection, cell fusion and ‘enucleation’ has yet to be investigated.

SCNT by nuclear transfer (NT; ‘Dolly’ approach) (Wilmut et al., 419 NATURE 583-7 (2002); Polejaeva et al., 407 NATURE 86-90 (2000); and Campbell et al., 380 NATURE 64-6 (1996)) and by ICNI (Honolulu method) (Wakayama et al., 394 NATURE 369-74 (1998) and Dominko et al., 1 CLONING 143-152 (1999)) both hold promise for propagating identical primates, but previously unanticipated biological hurdles, found only in primates, exist. Furthermore, crucial investigations regarding human embryonic stem cell potentials are investigated with non-human primates. Sets of genetically identical primate offspring would be invaluable for biomedical and behavioral investigations, yet none have been born yet. Therefore, the present invention provides reliable and effective methods for propagating identical and transgenic animals, and specifically primates. Furthermore, the present invention also provides various methodologies and molecular components that may be used for correcting mitotic spindle defects associated with NT.

### SUMMARY OF THE INVENTION

The present invention is directed to various methodologies to make NT a practical procedure for animals, and specifically primates. Furthermore, the methods and molecular components provided by the present invention provide a practical means for producing embryos with desired characteristics. In one embodiment, the methodology of the present invention comprises introducing nuclei nuclei with desired characteristics along with one or more molecular components into an egg, for example, an enucleated egg, culturing the egg to produce a viable embryo, transferring the embryo to the oviducts of a female, and producing a cloned animal.

In a specific embodiment, nuclei with desired characteristics may be obtained by selection or by design and transferred into eggs, for example, enucleated eggs. In a particular embodiment, normally occurring nuclei may be selected for genetic compatibility or complementarity to a host or may be derived or engineered from donors with desirable characteristics. In another embodiment of the present invention, the desired characteristics may be linked to a specific disease or disorder. In particular, the disease or disorder may comprise cardiovascular disease, neurological disease, reproductive disorder, cancer, eye disease, endocrine disorder, pulmonary disease, metabolic disorder, autoimmune disorder, and aging. Selected nuclei may be introduced into eggs along with molecular components comprising centrosomal components normally present in sperm centrosomes. In another embodiment, the molecular components comprise mitotic motor proteins and centrosome proteins, such as kinesins (e.g., HSET) and NuMA, respectively.

In particular, the methods of the present invention may comprise double nuclear transfer; meiotic spindle collapse, maternal DNA removal, and recovery; pronuclear removal after NT and fertilization or artificial activation; and cytoplasmic transfer or ooplasm supplementation.

In another embodiment of the present invention, the animal may be a mammal, bird, reptile, amphibian, or fish. In another aspect of this method, the animal may be a non-human primate, and in particular, a monkey. In an alternate aspect of this method, the animal may be a primate, and in particular, a human. In a particular embodiment of the present invention, the animal may be transgenic.

In a specific embodiment of the present invention, preimplantation genetic diagnosis may be performed on a blastomere isolated from the embryo prior to transfer to the oviduct of a female surrogate. The methods used for this preimplantation genetic diagnosis include polymerase chain reaction (PCR), fluorescence in situ hybridization (FISH), single-strand conformational polymorphism (SSCP), restriction fragment length polymorphism (RFLP), primed in situ labeling (PRINS), comparative genomic hybridization (CGH), single cell gel electrophoresis (COMET) analysis, heteroduplex analysis, Southern analysis and denatured gradient gel electrophoresis (DGGE) analysis.

Also within the scope of the present invention is the production of embryonic stem cells and transgenic embryonic stem cells generated by the methods of the present invention. In a specific embodiment, SCNT embryos are used to produce clonal offspring and the isolated blastomeres are used to produce an embryonic stem cell line. In a further embodiment, the SCNT embryos are transgenic, and these SCNT transgenic embryos are used to produce clonal transgenic offspring and the isolated transgenic blastomeres are used to produce transgenic embryonic stem cell lines.

The present invention also relates to methods of producing embryonic stem cells whereby blastomeres are dissociated from embryos and these cells are then cultured to produce stem cell lines. In a specific embodiment, the methods described herein are used to produce primate embryonic stem cells. In another aspect of the invention, the methods described herein are used to produce transgenic embryonic stem cells including, for example, transgenic primate embryonic stem cells.

The present invention is also directed to embryonic stem cells produced by the methods described herein. In a particular embodiment, the embryonic stem cells are primate embryonic stem cells. In a further embodiment, the embryonic stem cells are transgenic including, for example, transgenic primate embryonic stem cells. In yet another embodiment, the transgenic embryonic stem cells are human transgenic embryonic stem cells.

The present invention also relates to methods for preimplantation genetic diagnosis of an embryo. In a specific embodiment, blastomeres are dissociated from an embryo and genetic analysis is performed on a single blastomere. In a further embodiment of the present invention, the remaining blastomeres are cultured to an embryonic stage and subsequently implanted in a female surrogate. The methods used for the genetic analysis of the blastomere include PCR, FISH, SSCP, RFLP, PRINS, CGH, COMET analysis, heteroduplex analysis, Southern analysis, and DGGE analysis.

### BRIEF DESCRIPTION OF THE FIGURES

FIG. 1 provides a schematic illustration of the manipulations and events that occur during SCNT. The steps include enucleation or metaphase-II arrested meiotic spindle removal, somatic cell selection and preparation, nuclear transfer (NT) or intracytoplasmic nuclear injection (ICNI), wound healing and drug recovery from both spindle removal and nuclear introduction, and oocyte activation.

FIGS. 2A-2G illustrate that faulty mitotic spindles produce aneuploid embryos after primate NT. FIG. 2A illustrates a defective NT mitotic spindle with misaligned chromosomes centrosomal NuMA at meiosis. FIG. 2B illustrates a defective NT mitotic spindle with misaligned chromosomes centrosomal NuMA at mitosis. FIG. 2C illustrates that a defective NT mitotic spindle with misaligned chromosome centrosomal NuMA does not occur at NT mitosis. FIG. 2D illustrates that centrosomal kinesin HSET is also missing after NT. FIG. 2E illustrates that centromeric Eg5 is not missing after NT. FIG. 2F illustrates that bipolar mitotic spindles are with aligned chromosomes and centrosomal NuMA after NT into fertilized eggs. FIG. 2G provides DNA microtubule, NuMA, and kinesin imagining.

FIGS. 3A-3R provide a schematic illustration of manipulations and events that occur during therapeutic cloning. These steps, which are described herein, generally include oocyte collection, enucleation, nuclear transfer, activation, cell division and differentiation, and transfer to the patient.

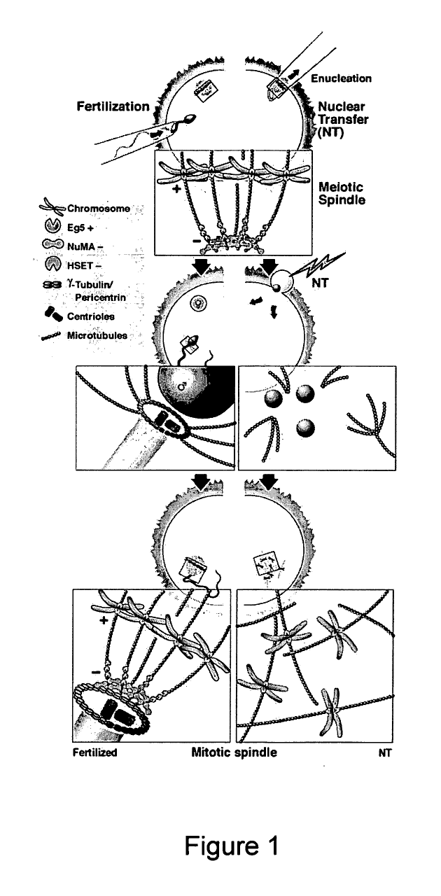

### DETAILED DESCRIPTION OF THE INVENTION

It is understood that the present invention is not limited to the particular methodology, protocols, and reagents, etc. described herein, as these may vary. It is also to be understood that the terminology used herein is used for the purpose of describing particular embodiments only, and is not intended to limit the scope of the present invention. It must be noted that as used herein and in the appended embodiments, the singular forms “a,” “an,” and “the” include plural reference unless the context clearly dictates otherwise.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

### Definitions

For convenience, the meaning of certain terms and phrases employed in the specification, examples, and appended embodiments are provided below.

The term “animal” includes all vertebrate animals such as mammals (e.g., rodents, mice and rats), primates (e.g., monkeys, apes, and humans), sheep, dogs, rabbits, cows, pigs, amphibians, reptiles, fish, and birds. It also includes an individual animal in all stages of development, including embryonic and fetal stages.

The term “primate” as used herein refers to any animal in the group of mammals, which includes, but is not limited to, monkeys, apes, and humans.

The term “totipotent” as used herein refers to a cell that gives rise to all of the cells in a developing cell mass, such as an embryo, fetus, and animal. In specific embodiments, the term “totipotent” also refers to a cell that gives rise to all of the cells in an animal. A totipotent cell can give rise to all of the cells of a developing cell mass when it is utilized in a procedure for creating an embryo from one or more nuclear transfer steps. An animal may be an animal that functions ex utero. An animal can exist, for example, as a live born animal. Totipotent cells may also be used to generate incomplete animals such as those useful for organ harvesting, e.g., having genetic modifications to eliminate growth of a head, or other organ, such as by manipulation of a homeotic gene.

The term “totipotent” as used herein is to be distinguished from the term “pluripotent.” The latter term refers to a cell that differentiates into a sub-population of cells within a developing cell mass, but is a cell that may not give rise to all of the cells in that developing cell mass. Thus, the term “pluripotent” can refer to a cell that cannot give rise to all of the cells in a live born animal.

The term “totipotent” as used herein is also to be distinguished from the term “chimeric” or “chimera.” The latter term refers to a developing cell mass that comprises a sub-group of cells harboring nuclear DNA with a significantly different nucleotide base sequence than the nuclear DNA of other cells in that cell mass. The developing cell mass can, for example, exist as an embryo, fetus, and/or animal.

The term “embryonic stem cell” as used herein includes pluripotent cells isolated from an embryo that may be maintained, for example, in in vitro cell culture. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells and their uses are well known to a person of skill in the art. See, e.g., U.S. Pat. No. 6,011,197 and WO 97/37009, entitled “Cultured Inner Cell Mass Cell-Lines Derived from Ungulate Embryos,” Stice and Golueke, both of which are incorporated herein by reference in their entireties, including all figures, tables, and drawings, and Yang & Anderson, 38 THERIOGENOL. 315-35 (1992).

For the purposes of the present invention, the term “embryo” or “embryonic” as used herein includes a developing cell mass that has not implanted into the uterine membrane of a maternal host. Hence, the term “embryo” as used herein can refer to a fertilized oocyte, a cybrid, a pre-blastocyst stage developing cell mass, and/or any other developing cell mass that is at a stage of development prior to implantation into the uterine membrane of a maternal host. Embryos of the invention may not display a genital ridge. Hence, an “embryonic cell” is isolated from and/or has arisen from an embryo.

An embryo can represent multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

The term “fetus” as used herein refers to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species. The term “fetal cell” as used herein can refer to any cell isolated from and/or arisen from a fetus or derived from a fetus. The term “non-fetal cell” is a cell that is not derived or isolated from a fetus.

The term “inner cell mass” as used herein refers to the cells that gives rise to the embryo proper. The cells that line the outside of a blastocyst are referred to as the trophoblast of the embryo. The methods for isolating inner cell mass cells from an embryo are well known to a person of ordinary skill in the art. See, Sims & First, 91 PROC. NATL. ACAD. SCI. USA 614347 (1994) and Keefer et al., 38 MOL. REPROD. DEV. 264-268 (1994). The term “pre-blastocyst” is well known in the art.

A “transgenic embryo” refers to an embryo in which one or more cells contain heterologous nucleic acid introduced by way of human intervention. The transgene may be introduced into the cell, directly or indirectly, by introduction into a precursor of the cell, by way of deliberate genetic manipulation, or by infection with a recombinant virus. In the transgenic embryos described herein, the transgene causes cells to express a structural gene of interest. However, transgenic embryos in which the transgene is silent are also included.

The term “transgenic cell” refers to a cell containing a transgene.

The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration of genetic information, they are transgenic animals as well.

The term “gene” refers to a DNA sequence that comprises control and coding sequences necessary for the production of a polypeptide or precursor. The polypeptide can be encoded by a full length coding sequence or by any portion of the coding sequence so long as the desired enzymatic activity is retained.

The term “transgene” broadly refers to any nucleic acid that is introduced into the genome of an animal, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may be normally present in the nontransgenic genome but which one desires to have altered in expression, or which one desires to introduce in an altered or variant form. The transgene may be specifically targeted to a defined genetic locus, may be randomly integrated within a chromosome, or it may be extrachromosomally replicating DNA. A transgene may include one or more transcriptional regulatory sequences and any other nucleic acid, such as introns, that may be necessary for optimal expression of a selected nucleic acid. A transgene can be coding or non-coding sequences, or a combination thereof. A transgene may comprise a regulatory element that is capable of driving the expression of one or more transgenes under appropriate conditions.

The phrase “a structural gene of interest” refers to a structural gene, which expresses a biologically active protein of interest or an antisense RNA, for example. The structural gene may be derived in whole or in part from any source known to the art, including a plant, a fungus, an animal, a bacterial genome or episome, eukaryotic, nuclear or plasmid DNA, cDNA, viral DNA, or chemically synthesized DNA. The structural gene sequence may encode a polypeptide, for example, a receptor, enzyme, cytokine, hormone, growth factor, immunoglobulin, cell cycle protein, cell signaling protein, membrane protein, cytoskeletal protein, or reporter protein (e.g., green fluorescent protein (GFP), β-galactosidase, luciferase). In addition, the structural gene may be a gene linked to a specific disease or disorder such as a cardiovascular disease, neurological disease, reproductive disorder, cancer, eye disease, endocrine disorder, pulmonary disease, metabolic disorder, autoimmune disorder, and aging.

A structural gene may contain one or more modifications in either the coding or the untranslated regions which could affect the biological activity or the chemical structure of the expression product, the rate of expression, or the manner of expression control. Such modifications include, but are not limited to, mutations, insertions, deletions, and substitutions of one or more nucleotides. The structural gene may constitute an uninterrupted coding sequence or it may include one or more introns, bound by the appropriate splice junctions. The structural gene may also encode a fusion protein.

Primates identical in both nuclear and cytoplasmic components represent ideal scientific models, for example, for preclinical investigations on the genetic and epigenetic basis of diseases. Here, the present invention relates to producing genetically identical primates as twin and higher-order multiples by using SCNT. Further, the present invention contemplates several methods for correcting dysfunctional reproductive potential in human and non-human primates and therapeutic value of cells and tissue derived from embryos after application of NT technology. For NT to be effective, the introduced diploid nucleus from a somatic or embryonic nucleus should be capable of condensing and aligning their duplicated chromosomes on a functional bipolar spindle apparatus at first mitosis. This may be followed by accurate segregation of the aligned chromosomes at the end of first division. The assembly of a functional bipolar spindle is, in turn, reliant on the two critical events: (i) the cell's microtubule organizing center (i.e., the somatic or embryonic centrosome) introduced during nuclear transfer which nucleates the spindle microtubules after nuclear envelope breakdown; and (ii) the action of a set of structural components (i.e., including nuclear mitotic apparatus protein (e.g., NuMA) and molecular motor proteins (including kinesin motor proteins)), which are largely contributed by the egg cell, and which crosslink, organize and shape the bipolar spindle apparatus for aligning and segregating the duplicated chromosomes. Previously, there has been no evaluation of the centrosomes or structural/molecular motor proteins role in bipolar spindle assembly after nuclear transfer. The present invention illustrates that dysfunctional centrosomes as well as missing NuMA and HSET kinesin result in mitotic multipolar spindles with misaligned chromosomes and aneuploid embryos after nuclear transfer.

The following embodiments of the present invention relate to various techniques for correcting mitotic spindle defects associated with NT. The methodologies provided by the present invention are capable of evaluating mechanisms for potential nuclear transfer failures related to first mitotic errors, previously elusive of efficient detection.

There is a direct correlation between reproduction and the ability to assemble bipolar mitotic spindles, which are responsible for accurately and faithfully segregating the duplicated chromosomes. Generally, in primates, the sperm contributes the centrioles, which are critical to the assembly of a functional centrosome. Following centrosome assembly, the centrosome participates in the assembly of the first mitotic spindle microtubules. The oocyte, in contrast, contributes various motor proteins, such as members of the kinesin superfamily and dynein, which coalesce on the mitotic spindle microtubules. The function of the various motor proteins is to participate in and maintain the assembly of the bipolar mitotic spindle.

The mitotic spindles are essential to the production of viable human and non-human primate embryos. It was unanticipated that spindle organization and accurate segregation of chromosomes would depend on these molecules. The necessity for these components is demonstrated by the non-viability of embryos prepared by NT that lack structural or motile molecules from the sperm centrosome. Therefore, spindle-organizing principles that are present in sperm may be required to produce useful success rates of NT and the practical production of embryos intended for producing cells, tissues or animals with selected characteristics.

The present invention contemplates that the introduction of centrosomal components may correct mitotic spindle defects associated with NT. Furthermore, the present invention provides some of the key components needed for the correction of mitotic spindle defects. In particular, these components may include, but are not limited to, NuMA and HSET kinesin.

The present invention also contemplates various methodologies that would make NT a practical procedure for animals, and specifically human or non-human primates. In particular, nuclei with desired characteristics would be obtained by selection or by design and transferred into eggs. Normally occurring nuclei may be selected for genetic compatibility or complementarity to a host or may be derived or engineered from donors with desirable characteristics. Selected nuclei would be introduced into eggs along with components normally present in sperm centrosomes. The addition of centrosomal components may be necessary for the production of viable embryos. The utility of these methods and molecules provided by the present invention creates a practical means for producing embryos with desired characteristics.

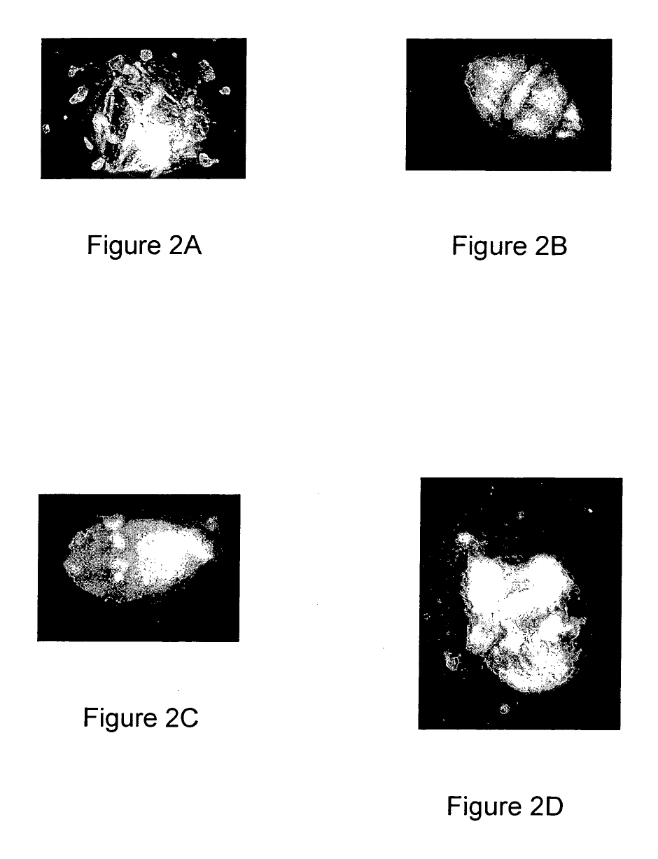

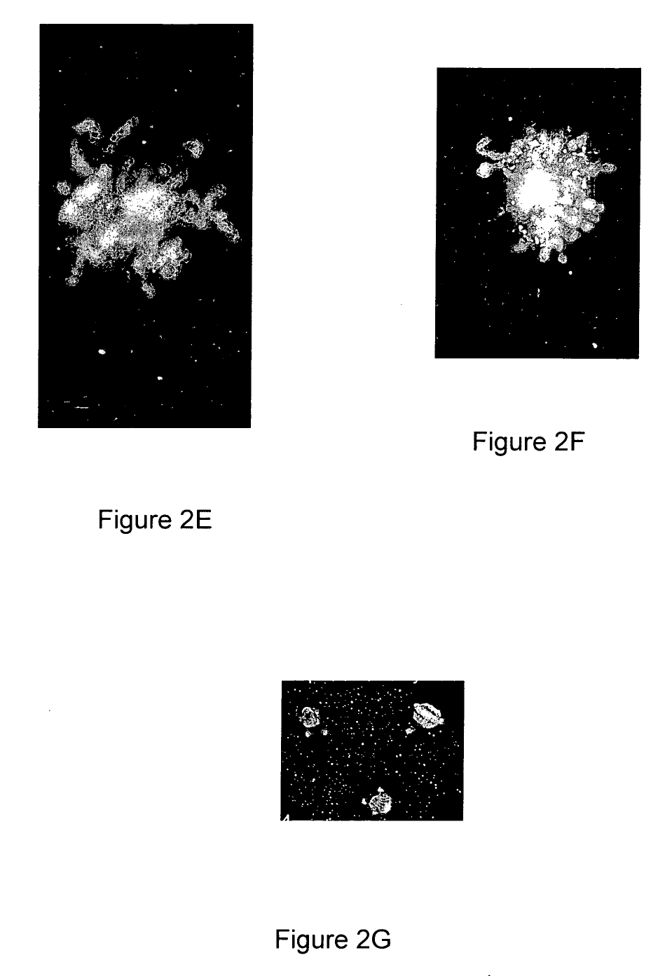

A specific embodiment of the present invention relates to the pronuclear removal after SCNT and fertilization (ICS/NI-2PN). FIGS. 2A-2G demonstrate the feasibility and benefit of pronuclear removal after SCNT and fertilization (ICS/NI-2PN). ICNI without prior oocyte enucleation is followed by fertilization. The somatic cell may be introduced distal from the first polar body allowing a geographical separation between the female pronucleus and the diploid nucleus. The sperm may be identified either by prelabeling its mitochondria with, for example, the vital dye MitoTracker or by imaging the incorporated sperm tail. At first interphase, the two pronuclei are extracted and the reprogrammed and remodeled somatic nucleus resides in the sperm-activated oocyte with the full complement of ooplasmic proteins restored after second meiotic completion. For a discussion of nuclear programming, see generally Wakayama et al., 5(3) CLONING AND STEM CELLS 181-89 (2003); Dinnyes et al., 4(1) CLONING AND STEM CELLS 81-90 (2002); and Jaenisch et al., 4(4) CLONING AND STEM CELLS 389-96 (2002).

Additionally, the present invention also contemplates a second NT so that the somatic nucleus may be reprogrammed and remodeled as during NT (double NT). However, the nucleus may be transferred the next day into another egg that had been fertilized by intracytoplasmic sperm injection (ICSI) previously. The male and female pronuclei (sperm and egg nuclei, respectively) of the zygote may be removed by micromanipulation. The second nuclear transfer, from the first interphase NT into the now enucleated zygote, inserts the reprogrammed somatic diploid nucleus into an interphase cytoplasm that has been activated by the sperm and contains all the ooplasmic constituents previously sequestered on the meiotic spindle. Normally, the spindle-associated motors are returned to the ooplasm after second polar body formation, and with the double NT strategy, they are in full complement. Double NT affords several advantages, including its successful application during SCNT in pigs. However, it requires twice the number of eggs and many more demanding intricate procedures, including pronuclear extraction coupled by interphase nuclear transfer.

Another embodiment of the present invention contemplates meiotic spindle collapse using reversible microtubule disassembly with either nocodazole or cold to reduce or eliminate the spindle microtubules. Dynamic DNA imaging identifies the meiotic chromosomes so they can be extracted without discarding the motor or centrosome molecules. Spindle collapse promises an efficient improvement, specifically, if oocyte recovery is complete and rapid.

Moreover, another embodiment of the present invention envisions using ooplasmic supplementation either by ooplast electrofusion (SCNT+OF) or microinjection (cytoplasmic transfer and ICNI; CT+ICNI), which has been used for bovine cloning and clinical ART (CT) (Barritt et al., 5 MOL. HUM. REPROD. 927-33 (1999)). Here additional ooplasm, from oocytes of the same clutch, supplements that lost during enucleation. An alternative embodiment may use the ooplasm from cold or nocodazole-recovery oocytes, for example, if the complete recovery within the same oocyte proves difficult. Ooplasmic supplementation has succeeded already in humans and cattle, and is particularly straightforward when combined with ICNI (i.e., ICNI+CT), just like the clinical ICSI+CT. ICS/NI-2PN (i.e., ICNI, ICSI and then pronuclear removal) uses half the oocytes of double NT. It demands a second day enucleation, but uses natural activation and avoids both cytochalasin and spindle disruption.

The present invention has several important benefits for the biomedical research community. By expanding animal and specifically primate reproduction to include transgenic and SCNT capabilities, the utility of this model for essential and urgent pre-clinical investigations may be greatly enhanced. SCNT may find extraordinary applications, were it developed as a reliable, routine approach for propagating invaluable primate models. Notwithstanding the technologies routinely available for creating rodent models for various diseases, many serious human disorders are not appropriately studied in these lower mammals. The production of transgenic primates as the most clinically relevant models for human diseases might well be critical for the entire clinical research community. Furthermore, the combination of these approaches might even result in reliable and efficient applications for propagating invaluable transgenic primates as research models. Finally, the promise of safe and effective gene therapy protocols cannot be fully realized until an appropriate system for investigation is found to fill the gap between transgenic mice and seriously ill patients. Consequently, there are strong justifications for developing reliable and effective methods for creating genetically modified primates, specifically, human and non-human primates. Thus, the present invention may have clinical and investigative applications which include, but are not limited to, cell therapy (neural, brain, and spinal stem cell applications, liver stem cell applications, pancreas stem cell applications, cardiac stem cell applications, renal stem cell applications, blood stem cell applications, retinal stem cell applications, diabetes-stem cell applications, orthopedics-stem cell applications, identical primate models for research, drug discovery, embryonic stem cells for drug discovery), pharmaceutical and medical devices (including animal models of disease for drug discovery and testing, pharmacological target identification, drug discovery, drug efficacy testing, biocompatibility of medical devices), agriculture, rare and endangered species, and toxicology evaluation.

Furthermore, the present invention also relates to methods of using embryonic stem cells and transgenic embryonic stem cells to treat human diseases. Specifically, the methods for clonal propagation of primates, specifically, human or non-human primates, described in the present invention, may also be used to create embryonic stem cells and transgenic embryonic stem cells.

Cells from the inner cell mass of an embryo (i.e., blastocyst) may be used to derive an embryonic stem cell line, and these cells may be maintained in tissue culture (see, e.g., Schuldiner et al., 97 PROC. NATL. ACAD. SCI. USA 11307-12 (2000); Amit et al., 15 DEV. BIOL. 271-78 (2000); U.S. Pat. No. 5,843,780; and U.S. Pat. No. 5,874,301). In general, stems cells are relatively undifferentiated, but may give rise to differentiated, functional cells. For example, hemopoietic stem cells may give rise to terminally differentiated blood cells such as erythrocytes and leukocytes.

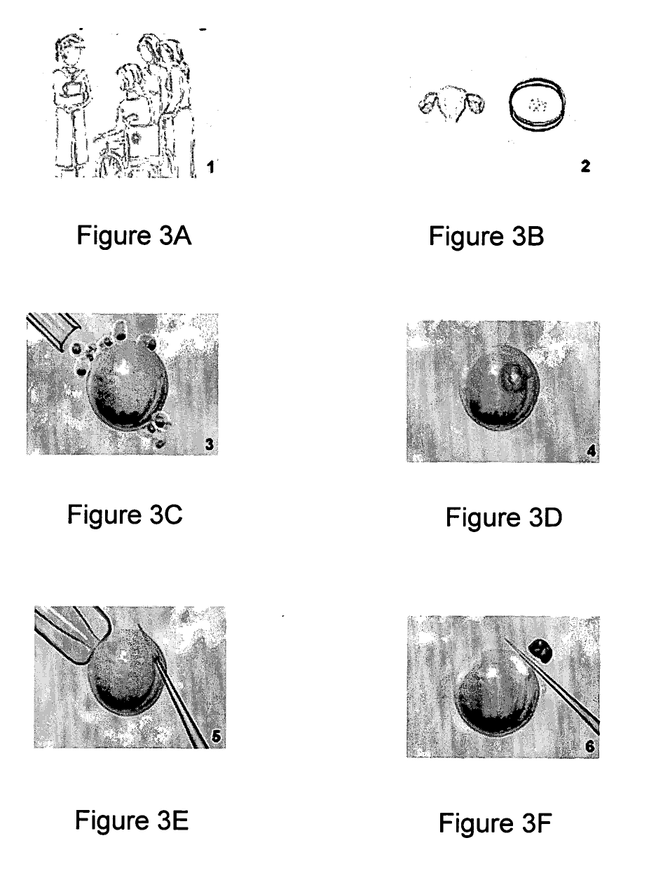

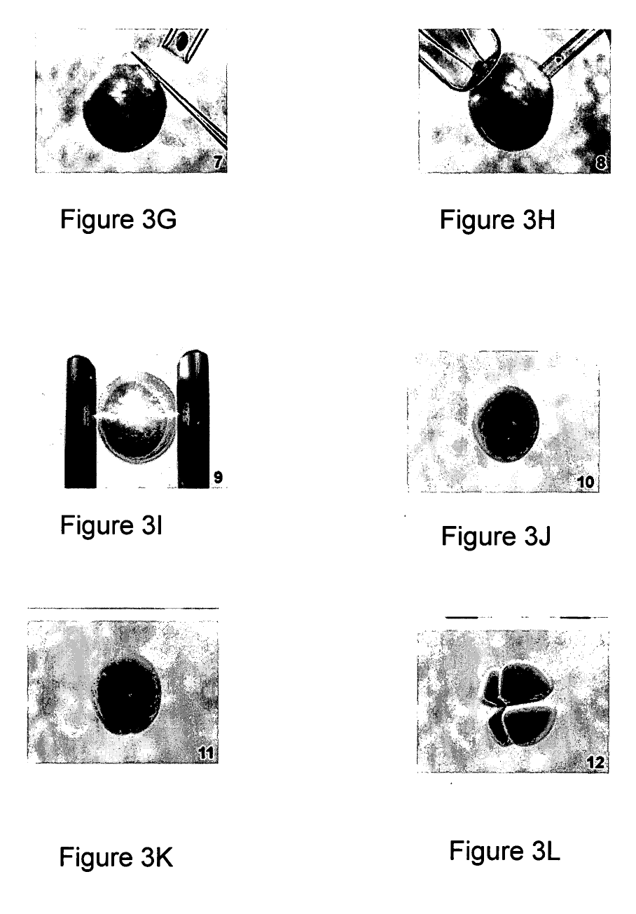

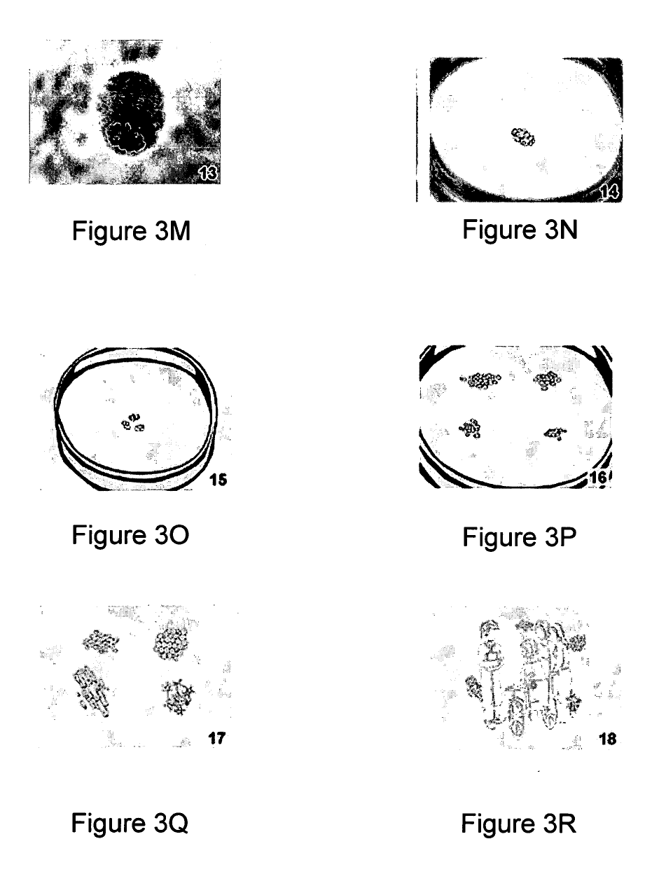

FIG. 3 provides the basic outline of such procedures, specifically, embryonic stem cells can grow into new nerves to heal injuries in a patient, such as spinal damage (FIG. 3A). Eggs are removed from the patient's ovaries (FIG. 3B) and placed in a petri dish. Cells that cling to the egg are removed (FIG. 3C). Inside the egg is the nucleus (Figure ED), which is removed in order to make a cloned embryo. For example, the egg is pierced with a fine needle or pipette (FIG. 3E), gently squeezed or aspirated to expel the nucleus (FIG. 3F), and the nucleus is removed (FIG. 3G). One of the cells removed from the egg previously (FIG. 3C) is injected into the egg (FIG. 3H). An electric current activates the egg (FIG. 3I). The genetic material of the injected cell guides the egg to develop (FIG. 3J). Cell division begins (FIG. 3K). Specifically, the genetic material is dividing so that it can be shared equally among the two new cells. The cells have divided again, to the four-cell stage (FIG. 3L). Many cell divisions later an area called the inner cell mass, which contains stem cells, is visible (FIG. 3M). The stem cells are removed and placed in a growth medium (FIG. 3N). The stem cells grow into colonies (FIG. 3O), which can be divided and grown repeatedly, resulting in millions of stem cells (FIG. 3P). These cells can grow into any tissue in the primate body, for example, muscle, nerve, pancreas, and bone cells (FIG. 3Q). Specialized cells may be placed back in a human patient at the site of injury or disease, so that new, working cells grow (FIG. 3R).

Using the methods of the present invention, transgenic primate embryonic stem cells may also be produced which express a gene related to a particular disease. For example, transgenic primate embryonic cells may be engineered to express tyrosine hydroxylase, which is an enzyme involved in the biosynthetic pathway of dopamine. In Parkinson's disease, this neurotransmitter is depleted in the basal ganglia region of the brain. Thus, transgenic primate embryonic cells expressing tyrosine hydroxylase may be grafted into the region of the basal ganglia of a patient suffering from Parkinson's disease and potentially restore the neural levels of dopamine (see, e.g., Bankiewicz et al., 144 EXP. NEUROL. 147-56 (1997)). The methods described in the present invention, therefore, may be used to treat numerous human diseases and disorders (see, e.g., Rathjen et al., 10 REPROD. FERTIL. DEV. 31-47 (1998); Guan et al., 16 ALTEX 135-41 (1999); Rovira et al., 96 BLOOD 4111-117 (2000); Muller et al., 14 FASEB J. 2540-48 (2000)).

Other objectives, features, and advantages of the present invention will become apparent from the following specific examples. The examples, while indicating specific embodiments of the invention, are provided by way of illustration only. Accordingly, the present invention also includes those various changes and modifications within the spirit and scope of the invention that may become apparent to those skilled in the art from this detailed description.

### EXAMPLES

### Example 1

### Oocyte Collection and Nuclear Transfer

Procedures for superstimulation, oocyte staging, and fertilization of rhesus eggs have been described by Hewitson et al. (77 FERTIL. STERIL. 794-801 (2002)). Enucleation of mature unfertilized oocytes, exposed briefly to TALP-Hepes with 7.5 μg/ml cytochalasin D (CCD) and 1 μg/ml Hoechst 33342 DNA dye (Sigma Chemical Co., St. Louis, Mo.), was performed using a 30 μm ID pipette (Humagen, Charlottsville, Va.) to aspirate the first polar body and underlying cytoplasm, including the second mitotic spindle (Meng et al., 57 BIOL. REPROD. 454-9 (1997)). Maternal chromatin removal was confirmed by Hoechst imaging. For embryonic cell nuclear transfer (ECNT), donor blastomeres were isolated from 4- to 32-cell embryos after zona pellucida removal with 0.5% pronase (Boehringer Mannheim) and culturing in Ca²⁺- and Mg²⁺-free TALP-Hepes with 1 mM each EDTA and EGTA. A single blastomere was pipetted into the perivitelline space of an enucleated oocyte and, after a 30-60 min. recovery, fused with two DC pulses (1.2 kV/cm, 30 μsec) using a BTX Cell Manipulator 2001 (Genentronics, Inc., San Diego, Calif.) in 0.3 M sorbitol, 0.1 mM calcium-acetate, 0.1 mM magnesium acetate and 0.5 mg/ml FFA-BSA (Sigma). ECNTs were performed using activation prior to blastomere fusion 2-4 hours later, as well as aged or interphase oocytes for recipient cytoplasts. For SCNT, enucleated oocytes were either directly injected with a single donor cell or fused after transfer of a donor cell under the zona pellucida. The cell nuclear donor source included dissociated granulosa cells, endothelial cells collected from rhesus umbilical cords, isolated, cultured ICM cells derived from rhesus blastocysts (2-3 passages), and primary rhesus fibroblast cell lines.

Activation Protocols. NT constructs were activated between 1-4 hours after cell fusion to enable nuclear reprogramming either by microinjection of 120-240 pg ml-1 rhesus sperm extract prepared in 120 mM KCl, 20 mM HEPES, 100 μM EGTA, 10 mM sodium glycerolphosphate (Wilmut et al., 419 Nature 583-87 (2002)) and sterilized through a 0.22 μm SpinX filter (Costar, Cambridge, Mass.) or by the sequential treatment of about 5 μM to about 10 μM ionomycin (5 min.; room temperature) and 1.9 mM DMAP for 4 hours (Chanet al., 287 SCIENCE 317-19 (2000)). After DMAP, eggs were washed extensively and cultured in TALP. FertClones were produced by the fusion of a cytoplast containing removed maternal chromatin with an enucleated oocyte and then fertilized by ICSI 2-3 hours later. All were cultured in TALP for 24 hours and then buffalo rat liver cell (BRL) monolayers in CMRL medium.

Microinjection. Antibody inhibition studies were performed using 9-μm micropipettes (Humagen) front-loaded with the primary antibody. Between 4-6% of the egg volume (˜700 pl) was microinjected with antibodies at 2-10 mg ml-1. Final antibody concentrations were at between 70-350 pg total Ig protein per oocyte. For imaging microinjected oocytes and eggs, the primary antibody was omitted.

Embryo Transfer. For embryo transfer, surrogate rhesus females were selected on the basis of serum estradiol and progesterone levels. Pregnancies were ascertained by endocrinological profiles and fetal ultrasound performed between days 24-30 (Wilmut et al., 385 NATURE 810-3 (1997)).

Imaging. Methods for fixation and immunocytochemistry of oocytes and NTs were performed as described. Mitalipov et al., 66 BIOL. REPROD. 1449-55 (2002). Centrosomes were detected with g-tubulin or pericentrin polyclonal rabbit antibodies (gifts of Dr. T. Steams [Stanford] and S. Doxsey [U. of Mass], respectively). Thomson et al., 92 PROC. NATL. ACAD. SCI. USA 7844-48 (1995) and Thomson et al., 282 SCIENCE 1145-47 (1998). NuMA, HSET and Eg5 were detected using rabbit polyclonal antibodies as described. Wilmut et al. (1997), and Humpherys et al. (2002). Microtubules were immunolabeled with E7 mouse monoclonal antibody. Wilmut et al., 385 NATURE 810-3 (1997). Primary antibodies were detected using Alexa secondary antibodies (Alexa-546 goat anti-mouse IgG; Alexa-488 goat anti-rabbit IgG; Molecular Probes, Eugene, Org.) for observation by confocal microscopy. Each primary and secondary antibody was applied for 40 min at 37° C.; rinses performed with PBS+0.1% Triton. DNA was fluorescently detected with 5 μg/ml Hoechst 33342 (Sigma) and 1 μM Toto-3 (Molecular Probes) added to the penultimate rinse. Coverslips were mounted in Vectashield antifade (Vector Labs, CA). Controls included nonimmune and secondary antibodies alone, which did not detect spindle microtubules or centrosomes. Slides were examined using conventional immunofluorescence and laser-scanning confocal microscopy. Conventional fluorescence microscopy was performed first using a Nikon Eclipse E1000 microscope with high numerical aperture objectives. Data was recorded digitally using a cooled CCD camera (Hamamatsu Instruments Inc., Japan) using Metamorph software (Universal Imaging, West Chester, Pa.). Laser-scanning confocal microscopy was performed using a Leica SP-2 equipped with Argon and Helium-Neon lasers for the simultaneous excitation of Alexa-conjugated secondary antibodies and Toto-3 DNA stain.

Immunoblotting. Murine oocytes and human ovarian protein extracts (Clontech, Inc., Palo Alto, Calif.) were separated on linear gradient SDS-PAGE gels and Western blots as described by Humpherys et al. (2002).

### Example 2

### Production of Embryonic Stem Cells

ES cells are established from embryos by the following method. Following SCNT, 2-4 blastomeres are cultured in a microwell, which contains a monolayer of feeder cells derived from mouse embryonic fibroblasts (MEF), or primate embryonic fibroblasts (PEF), either human or non-human (Richards et al., 21(5) Stem Cells 546-56 (2003); Richards et al., 20 Nature Biotech. 933-36 (2002)). The remaining embryo is then transferred to an empty zona for embryo reconstruction as described in Example 1. This co-culture system for isolating and culturing an ES cell line is well known in the art (see, e.g., Thomson et al., 92 Proc. Natl. Acad. Sci. USA 7844-48 (1995); Ouhibi et al., 40 Mol. Reprod. Dev. 311-24 (1995)). It has been suggested that the feeder cells provide growth factor-like leukemia inhibiting factor (LIF), which inhibits stem cell differentiation. The microwells contain 5-10 μl of culture medium (80% DMEM as a basal medium, 20% FBS, 1 mM β-mercaptoethanol, 1000 units/ml LIF, non-essential amino acids, and glutamine). The cells are then incubated at 37° C. with 5% CO2 and covered with mineral oil. Fresh medium is replaced everyday and the survival of blastomeres is determined by cell division. During the initial culture, cell clumps are dissociated mechanically until cell attachment to the MEF monolayer and colony formation is observed. The colonies are then passaged to a 4-well plate and subsequently to a 35 mm dish in order to expand the culture gradually until a stable cell line is established. In addition to the dissociated blastomere culture, the reconstructed embryos are also cultured until the blastocyst stage is reached. Hatch blastocysts or blastocysts without zonae are cultured on a MEF monolayer in a microwell as described above. Instead of dissociating the blastomeres, the blastocysts are allowed to attach to the MEF monolayer. Once the blastocysts attach to the MEF, the ICM cells are isolated mechanically and transferred to a fresh culture well. The embryonic cells are cultured as described above and expansion of the cells is continued until individual colonies are observed. Individual colonies are selected for clonal expansion. This clonal selection and expansion process continues until a clonal cell line is established.

Infection of unfertilized oocytes by a pseudotyped retroviral vector has been used successfully to produce a transgenic non-human primate. These methods are disclosed in co-pending U.S. patent applications Ser. No. 09/736,271 and Ser. No. 09/754,276, which are expressly incorporated herein by reference. The presence of the transgene was demonstrated in all tissues of the transgenic monkey, which suggests an early integration event has occurred, perhaps in the maternal chromosome prior to fertilization. To produce a transgenic embryonic stem cell line, the transgenic embryos produced by pseudotype infection are dissociated as described above in the clonal embryo production process. These split embryos are then used to produce clonal offspring or its embryonic counterpart is used to produce a transgenic embryonic stem cell line. Thus, the transgenic offspring and the transgenic embryonic stem cell line share the same genetic modification that was achieved at the oocyte stage.

Various modifications and variations of the described examples and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention, which are obvious to those skilled in related fields, are intended to be within the scope of the following embodiments.

What is claimed is:

1. A method comprising the steps of:
introducing nuclei along with one or more molecular components into an egg;
culturing said egg to produce a viable embryo;
transferring said embryo to the oviducts of a female; and
producing a cloned animal.

2. The method of claim 1, wherein said nuclei have desired characteristics.

3. The method of claim 2, wherein said desired characteristics are linked to a specific disease or disorder.

4. The method of claim 3, wherein said specific disease or disorder is selected from the group consisting of cardiovascular disease, neurological disease, reproductive disorder, cancer, eye disease, endocrine disorder, pulmonary disease, metabolic disorder, autoimmune disorder, and aging.

5. The method of claim 1, wherein said introducing step comprises performing SCNT.

6. The method of claim 5, further comprising the step of performing pronuclear removal after SCNT.

7. The method of claim 5, further comprising the step of performing a second nuclear transfer following said SCNT.

8. The method of claim 1, wherein said introducing step further comprises performing meiotic spindle collapse.

9. The method of claim 1, further comprising the step of performing ooplasmic supplementation following said introducing step.

10. The method of claim 9, wherein said ooplamsic supplementation is performed by ooplast electrofusion.

11. The method of claim 9, wherein said ooplasmic supplementation is performed by microinjection.

12. The method of claim 1, wherein said one or more molecular components comprise centrosomal components normally present in sperm centrosomes.

13. The method of claim 1, wherein said one or more molecular components comprise mitotic motor proteins and centrosome proteins.

14. The method of claim 13, wherein said mitotic motor proteins comprise kinesins.

15. The method of claim 14, wherein said kinesins comprise HSET kinesin.

16. The method of claim 13, wherein said centrosome proteins comprise NuMA.

17. The method of claim 1, wherein said animal is a primate.

18. The method of claim 17, wherein said animal is a non-human primate.

19. The method of claim 18, wherein said non-human primate is a monkey.

20. The method of claim 17, wherein said primate is a human.

21. The method of claim 1, wherein said viable embryo is transgenic.

22. The method of claim 1, further comprising the step of producing embryonic stem cells from said viable embryo.

23. The method of claim 22, wherein said embryonic stem cells are human and said viable embryo is human.

24. An animal produced by the method of claim 1.

25. The animal of claim 24, wherein said animal is a primate.

26. The animal of claim 25, wherein said primate is a non-human primate.

27. The animal of claim 25, wherein said primate is a human.

28. A method comprising the steps of:
introducing nuclei along with one or more molecular components into an egg;
culturing said egg to produce a viable embryo;
dissociating blastomeres from said embryo; and
culturing said blastomeres to produce stem cells.

29. The method of claim 28, wherein said nuclei have desired characteristics.

30. The method of claim 29, wherein said desired characteristics are linked to a specific disease or disorder.

31. The method of claim 30, wherein said specific disease or disorder is selected from the group consisting of cardiovascular disease, neurological disease, reproductive disorder, cancer, eye disease, endocrine disorder, pulmonary disease, metabolic disorder, autoimmune disorder, and aging.

32. The method of claim 28, wherein said introducing step comprises performing SCNT.

33. The method of claim 32, further comprising the step of performing pronuclear removal after said SCNT.

34. The method of claim 32, further comprising the step of performing a second nuclear transfer following said SCNT.

35. The method of claim 28, wherein said introducing step further comprises performing meiotic spindle collapse.

36. The method of claim 28, further comprising the step of performing ooplasmic supplementation following said introducing step.

37. The method of claim 36, wherein said ooplamsic supplementation is performed by ooplast electrofusion.

38. The method of claim 36, wherein said ooplasmic supplementation is performed by microinjection.

39. The method of claim 28, wherein said one or more molecular components comprise centrosomal components normally present in sperm centrosomes.

40. The method of claim 28, wherein said one or more molecular components comprise mitotic motor proteins and centrosome proteins.

41. The method of claim 40, wherein said mitotic motor proteins comprise kinesins.

42. The method of claim 41, wherein said kinesins comprise HSET kinesin.

43. The method of claim 40, wherein said centrosome proteins comprise NuMA.

44. The method of claim 28, wherein said stem cells are primate embryonic stem cells.

45. The method of claim 44, wherein said embryonic stem cells are transgenic primate embryonic stem cells.

46. The method of claim 45, wherein said transgenic embryonic stem cells are transgenic non-human primate embryonic stem cells.

47. An embryonic stem cell produced by the method of claim 28.

48. The embryonic stem cell of claim 47, wherein said embryonic stem cell is used for gene therapy.

49. The embryonic stem cell of claim 47, wherein said embryonic stem cell is used as a therapy for human disease.

50. A method comprising the steps of:
introducing nuclei along with one or more molecular components into an egg;
culturing said egg to produce a viable embryo; and
transferring said embryo to the oviducts of a female.

51. The method of claim 50, wherein said nuclei have desired characteristics.

52. The method of claim 51, wherein said desired characteristics are linked to a specific disease or disorder.

53. The method of claim 52, wherein said specific disease or disorder is selected from the group consisting of cardiovascular disease, neurological disease, reproductive disorder, cancer, eye disease, endocrine disorder, pulmonary disease, metabolic disorder, autoimmune disorder, and aging.

54. The method of claim 50, wherein said introducing step comprises performing SCNT.

55. The method of claim 54, further comprising the step of performing pronuclear removal after SCNT.

56. The method of claim 54, further comprising the step of performing a second nuclear transfer following said SCNT.

57. The method of claim 50, wherein said introducing step further comprises performing meiotic spindle collapse.

58. The method of claim 50, further comprising the step of performing ooplasmic supplementation following said introducing step.

59. The method of claim 58, wherein said ooplamsic supplementation is performed by ooplast electrofusion.

60. The method of claim 58, wherein said ooplasmic supplementation is performed by microinjection.

61. The method of claim 50, wherein said molecular components comprise centrosomal components normally present in sperm centrosomes.

62. The method of claim 50, wherein said molecular components comprise mitotic motor proteins and centrosome proteins.

63. The method of claim 62, wherein said mitotic motor proteins comprise kinesins.

64. The method of claim 63, wherein said kinesins comprise HSET kinesin.

65. The method of claim 62, wherein said centrosome proteins comprise NuMA.

66. The method of claim 50, wherein said viable embryo is transgenic.

67. The method of claim 50, further comprising the step of producing embryonic stem cells from said viable embryo.

68. The method of claim 67, wherein said embryonic stem cells are human and said viable embryo is human.

69. A method comprising the steps of:
introducing nuclei along with one or more molecular components into an egg;
culturing said egg to produce a viable embryo; and
dissociating blastomeres from said embryo.

70. The method of claim 69, wherein said nuclei have desired characteristics.

71. The method of claim 70, wherein said desired characteristics are linked to a specific disease or disorder.

72. The method of claim 71, wherein said specific disease or disorder is selected from the group consisting of cardiovascular disease, neurological disease, reproductive disorder, cancer, eye disease, endocrine disorder, pulmonary disease, metabolic disorder, autoimmune disorder, and aging.

73. The method of claim 69, wherein said introducing step comprises performing SCNT.

74. The method of claim 73, further comprising the step of performing pronuclear removal after said SCNT.

75. The method of claim 73, further comprising the step of performing a second nuclear transfer following said SCNT.

76. The method of claim 69, prior to said introducing step performing meiotic spindle collapse.

77. The method of claim 69, further comprising the step of performing ooplasmic supplementation following said introducing step.

78. The method of claim 77, wherein said ooplamsic supplementation is performed by ooplast electrofusion.

79. The method of claim 77, wherein said ooplasmic supplementation is performed by microinjection.

80. The method of claim 69, wherein said molecular components comprise centrosomal components normally present in sperm centrosomes.

81. The method of claim 69, wherein said molecular components comprise mitotic motor proteins and centrosome proteins.

82. The method of claim 81, wherein said mitotic motor proteins comprise kinesins.

83. The method of claim 82, wherein said kinesins comprise HSET kinesin.

84. The method of claim 81, wherein said centrosome proteins comprise NuMA.
